(19) United States
(12) Patent Application Publication
(10) Pub. No.: US 2025/0392273 A1
(43) Pub. Date: Dec. 25, 2025

(54) Systems and Methods for Improved Neurophysiological Monitoring

(71) Applicant: Cadwell Laboratories, Inc., Kennewick, WA (US)

(72) Inventors: Rose Rehfeldt, Kennewick, WA (US); Richard A. Villarreal, West Richland, WA (US); Ivan Amaya, Richland, WA (US); Ross Delvin, Kennewick, WA (US); Michael Batdorf, Benton City, WA (US); John A. Cadwell, Richland, WA (US)

(21) Appl. No.: 19/247,450

(22) Filed: Jun. 24, 2025

Related U.S. Application Data
(60) Provisional application No. 63/663,621, filed on Jun. 24, 2024.

(51) Int. Cl. H03F 3/45; A61B 5/31; A61B 5/378; A61B 5/38; H03K 19/0175

(52) U.S. Cl. CPC H03F 3/45071; A61B 5/31; A61B 5/378; A61B 5/38; H03K 19/017581; H03F 2200/375; H03F 2200/78; H03F 2203/45424; H03F 2203/45438

(57) ABSTRACT

A multi-modality intraoperative neurophysiological monitoring (IONM) system includes at least one amplifier integrated with a programmable switch matrix module configured to have up to 32 patient connected electrode inputs that are multiplexed to up to 24 amplifier channels. In some scenarios, a capacitor is positioned in series with electrode inputs of the at least one amplifier, such that the capacitor eliminates DC offset voltages from the electrode inputs while allowing neural AC signals to pass through to the at least one amplifier. In some scenarios, the switch matrix module includes a digital control loop configured to automatically eliminate offset voltage potential in patient connected electrodes. The switch matrix module further includes a field programmable gate array (FPGA) configured to automatically reset a plurality of control lines of one or more multiplexers of the switch matrix module.

### CROSS-REFERENCE

The present application relies on, for priority, U.S. Patent Provisional Application No. 63/663,621, titled “Systems and Methods for Improved Neurophysiological Monitoring” and filed on Jun. 24, 2024, which is herein incorporated by reference in its entirety.

### FIELD

The present specification relates generally to the field of neurophysiological monitoring. More specifically, the present specification relates to an amplifier with an integrated switch matrix that is configured to have an increased number of amplifier channels, supports automatic resetting of channel configuration, and optionally, addresses electrode offset voltage potential.

### BACKGROUND

Intraoperative neurophysiological monitoring (IONM) is directed towards identifying, mapping, and monitoring neural structures in accordance with their functions with a goal of preserving the structural integrity of those neural structures during physically invasive procedures such as surgery. In some methods, identifying, mapping and monitoring neural structures comprises applying electrical stimulation at or near an area where the target neural structures are believed to be located. Application of the electrical stimulation is transmitted through the nervous system structures to excite or depress the associated response(s) or function(s). For example, an electrical impulse is generated in the muscle(s), as a result of the excitation, that can be sensed using recording electrodes, thereby indicating presence and functionality of a neural structure to a surgeon.

Electroencephalography (EEG) is the neurophysiologic sensing and measurement of electrical activity of the brain by recording signals acquired from electrodes, which may be placed on the scalp, intracranially, on the surface of the brain, or within the brain tissue, and connected to an amplifier or recording device. The resulting traces are known as an electroencephalogram (EEG) and represent an electrical signal (postsynaptic potentials) from a large number of neurons.

Electrocorticography (ECoG) and stereoelectroencephalography (sEEG) are methods of intracranial EEG monitoring and cortical mapping that require high channel count recording and stimulating devices. Cortical stimulation mapping (CSM) is a type of electrocorticography that involves a physically invasive procedure and aims to localize the function of specific brain regions through direct electrical stimulation of the cerebral cortex. These systems use amplifiers capable of receiving input electrodes. Conventional high channel-count systems deploy multiple amplifier or recording devices as part of a sensing system and at least one neurostimulator as part of a stimulation system.

In a traditional IONM amplifier, two physical inputs (the location where leads are attached) are required to form one channel—an active-reference pair of inputs where “active” (input 1) minus “reference” (input 2) results in channel A. Therefore, a traditional 32 input amplifier provides 16 channels of data. If additional channels of data are needed, additional amplifiers must be added in parallel.

In an IONM amplifier, channel configuration is set by control lines. If the control lines are not set correctly, it may result in improper monitoring of a patient's biopotential signals. Because external electrical interference may disturb the control lines, standard design topologies that use latching multiplexers, such as crosspoint switches, or discrete digital latches will not indicate if the latches are disturbed. A direct connection of the control lines to a microcontroller is also not desirable since the microcontroller may add electrical noise to the control lines and cause noise to be added to the biopotential signal being switched by a multiplexer.

Additionally, electrodes placed on a patient exhibit an offset voltage potential between any two electrodes. The offset voltage potential may be on the order of 0.2 V to 1 V. This electrode voltage potential may vary slowly over time. An IONM amplifier must amplify the microvolt biopotential signal detected by the electrodes while rejecting the offset voltage potential. Currently, AC coupling is used to effectively eliminate offset voltage potential. While this approach offers clear benefits, it is important to note that it may also extend the amplifier's settling time following electrical disturbances such as those from electrocautery, as well as lengthen the recovery time after electrical stimulation (shock artifact). Alternatively, a DC-coupled solution with DC offset correction may be employed.

Accordingly, there is need for systems and methods that determine whether the channel configurations are scrambled or otherwise improper and automatically reset each configuration based on the determination. What is also needed are systems and methods that address electrode DC offset voltage potential.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses an electronic circuit adapted to operate as a switch matrix in an amplifier of an intraoperative neurophysiological monitoring (IONM) system, wherein the amplifier comprises a plurality of channels, the electronic circuit comprising: a microcontroller; a plurality of control lines in electrical communication with one or more multiplexers, wherein the plurality of control lines is adapted to configure each channel of a plurality of channels of the amplifier; a field programmable gate array (FPGA) in data communication with the microcontroller and the plurality of control lines, wherein the FPGA comprises an input register, a parallel bus latched at a readback register and at a first output register and a second output register, and a comparator, wherein the comparator is configured to receive first data of the first output register and second data of the second output register; and a logic module comprising a plurality of programmatic instructions that, when executed, cause the microcontroller to: write configuration data serially to the FPGA; receive an error flag from the comparator, wherein the error flag is indicative of the first data not matching the second data; and re-write, in response to the error flag, the configuration data to the FPGA.

Optionally, the logic module further comprises a plurality of programmatic instructions that, when executed, cause the microcontroller to convert the serially written configuration data to the parallel bus.

Optionally, the readback register is configured to allow readback of the configuration data serially by the microcontroller.

Optionally, the first output register is connected to external pins in electrical communication with the plurality of control lines. Optionally, the second output register is not connected to any external pins.

Optionally, the electronic circuit further comprises a high frequency noise detector in data communication with the microcontroller. Optionally, the logic module is configured to suspend re-writing of the configuration data when the high frequency noise detector indicates presence of noise, and configured to commence the re-writing of the configuration data only after a predefined time period without noise.

Optionally, the configuration data comprises values indicative of electrode input multiplexing, channel amplifier gain, and channel filter frequency.

Optionally, the switch matrix further comprises a digital control loop having an analog to digital converter (ADC) configured to digitize a signal at each channel of the plurality of channels of the amplifier to generate a first value indicative of an offset voltage potential, a digital to analog converter (DAC), and analog circuitry, wherein the ADC and DAC are in data communication with the microcontroller, and wherein the logic module further causes the microcontroller to: receive the first value; continue to increase a counter by incremental values until the first value exceeds a positive guard limit or continue to decrease the counter by decremented values until the first value exceeds a negative guard limit; determine a modulus value of the incremented or decremented counter value; adjust the DAC potential to a second value that is equal and opposite to the first value, if the modulus value exceeds an offset guard threshold; and apply the second value of the DAC potential to said each channel in order to nullify an effect of the offset voltage potential. Optionally, the switch matrix comprises a first multiplexer configured to allow a first input on its output line and a second multiplexer configured to allow a second input on its output line. Optionally, the switch matrix further comprises a differential amplifier, wherein the differential amplifier receives the first input and the second input in order to generate said each channel as a difference of the first input and the second input. Optionally, another adjustment of the DAC potential is suspended for a predefined period of time.

The present specification also discloses a method of automatically re-writing configuration data of a plurality of control lines of one or more multiplexers of a switch matrix module, wherein the switch matrix module is integrated into an amplifier of an intraoperative neurophysiological monitoring (IONM) system, and wherein the plurality of control lines configures a plurality of channels of the amplifier, the method comprising: writing configuration data serially to a field programmable gate array (FPGA) having an input register and a comparator, wherein the comparator receives first data of the first output register and second data of the second output register, and wherein the FPGA is in data communication with a microcontroller and the plurality of control lines; receiving an error flag from the comparator, wherein the error flag is indicative of the first data not matching the second data; and re-writing, in response to the error flag, the configuration data to the FPGA.

Optionally, the FPGA further comprises a parallel bus latched at a readback register and at a first output register and a second output register. Optionally, the method further comprises converting the serially written configuration data to the parallel bus. Optionally, the readback register allows readback of the configuration data serially by the microcontroller. Optionally, the first output register is connected to external pins used as the plurality of control lines. Optionally, the second output register is not connected to any external pins.

Optionally, the switch matrix module further comprises a high frequency noise detector in data communication with the microcontroller. Optionally, the re-writing of the configuration data is suspended when the high frequency noise detector indicates presence of noise, and wherein the re-writing commences only after a predefined time period of no noise.

Optionally, the configuration data comprises values indicative of electrode input multiplexing, channel amplifier gain, and channel filter frequency.

Optionally, the switch matrix module further comprises a digital control loop having an analog to digital converter (ADC) configured to digitize a signal at each channel of the plurality of channels of the amplifier to generate a first value indicative of an offset voltage potential, a digital to analog converter (DAC), and analog circuitry, wherein the ADC and DAC are in data communication with the microcontroller, and the method further comprises: receiving, from the ADC, a first value indicative of an offset voltage potential at said each channel; continuing to increase a counter by incremental values until the first value continues to exceed a positive guard limit or continuing to decrease the counter by decremented values until the first value continues exceed a negative guard limit; determining a function of the incremented or decremented counter value; adjusting the DAC potential to a second value that is equal and opposite to the first value, if an output of the function exceeds an offset guard threshold; and applying the second value of the DAC potential to said each channel in order to nullify an effect of the offset voltage potential. Optionally, the switch matrix module comprises a first multiplexer configured to allow a first input on its output line and a second multiplexer configured to allow a second input on its output line. Optionally, the switch matrix module further comprises a differential amplifier wherein the differential amplifier receives the first and second inputs in order to generate said each channel as a difference of the first and second inputs. Optionally, another adjustment of the DAC potential is suspended for a predefined period of time. Optionally, the function is a modulus value.

The present specification also discloses an intraoperative neurophysiological monitoring (IONM) system comprising: a base module; a power module, wherein the base module is electrically coupled to a computing device through the power module; an auditory and visual stimulator (AVX) module in electrical communication with the base module; at least one electrical stimulator in electrical communication with the AVX module and with the base module; at least one amplifier module in electrical communication with the base module; and at least one transcranial stimulator (TCS) extender in electrical communication with the base module.

Optionally, the intraoperative neurophysiological monitoring (IONM) system further comprises an identification (ID) chip in a connector at a patient end of a cable connected to the at least one amplifier module.

Optionally, the intraoperative neurophysiological monitoring (IONM) system further comprises an identification (ID) chip in a connector at a patient end of a cable connected to the at least one TCS extender.

The present specification also discloses an electronic circuit configured as a digital control loop within a programmable switch matrix module of an amplifier of an intraoperative neurophysiological monitoring (IONM) system, comprising: an analog to digital converter (ADC) configured to digitize a signal at a channel of the amplifier to generate a first value indicative of an offset voltage potential; a digital to analog converter (DAC), wherein the ADC and DAC are in data communication with a microcontroller; analog circuitry; and a logic module including a plurality of programmatic instructions which when executed cause the microcontroller to: receive the first value; continue to increase a counter by incremental value steps until the first value exceeds a positive guard limit or continue to decrease the counter by decremented value steps until the first value exceeds a negative guard limit; determine a modulus value of the incremented or decremented counter value; adjust the DAC potential to a second value that is equal and opposite to the first value, if the modulus value exceeds an offset guard threshold; and apply the second value of the DAC potential to the channel in order to nullify an effect of the offset voltage potential.

Optionally, the switch matrix module comprises a first multiplexer configured to allow a first input on its output line and a second multiplexer configured to allow a second input on its output line.

Optionally, the switch matrix module further comprises a differential amplifier, and wherein the differential amplifier receives the first input and the second input in order to generate the channel as a difference of the first input and the second input.

The electronic circuit of claim 18, wherein another adjustment of the DAC potential is suspended for a predefined period of time.

The present specification also discloses a method of using an electronic circuit to automatically adjust offset voltage potential signal in a channel of an amplifier of an intraoperative neurophysiological monitoring (IONM) system, wherein the electronic circuit is implemented within a programmable switch matrix module of the amplifier and includes an analog to digital converter (ADC), a digital to analog converter (DAC) and analog circuitry, the method comprising: receiving, from the ADC, a first value indicative of an offset voltage potential at the channel; continuing to increase a counter by incremental value steps until the first value continues to exceed a positive guard limit or continuing to decrease the counter by decremented value steps until the first value continues exceed a negative guard limit; determining a function of the incremented or decremented counter value; adjusting the DAC potential to a second value that is equal and opposite to the first value, if an output of the function exceeds an offset guard threshold; and applying the second value of the DAC potential to the channel in order to nullify an effect of the offset voltage potential.

Optionally, the switch matrix module comprises a first multiplexer configured to allow a first input on its output line and a second multiplexer configured to allow a second input on its output line.

Optionally, the switch matrix module further comprises a differential amplifier wherein the differential amplifier receives the first and second inputs in order to generate the channel as a difference of the first and second inputs.

Optionally, another adjustment of the DAC potential is suspended for a predefined period of time.

Optionally, the function is a modulus value.

The present specification also discloses an intraoperative neurophysiological monitoring (IONM) system comprising: a base module; a power module, wherein the base module is electrically coupled to a computing device through the power module; an auditory and visual stimulator (AVX) module in electrical communication with the base module; at least one electrical stimulator in electrical communication with the AVX module and with the base module; at least one amplifier module in electrical communication with the base module; and at least one transcranial stimulator (TCS) extender in electrical communication with the base module.

Optionally, the intraoperative neurophysiological monitoring (IONM) system further comprises an identification (ID) chip in a connector at a patient end of a cable connected to the at least one amplifier module.

Optionally, the intraoperative neurophysiological monitoring (IONM) system further comprises an identification (ID) chip in a connector at a patient end of a cable connected to the at least one TCS extender.

Further disclosed herein is an amplifier module for an intraoperative neurophysiological monitoring (IONM) system, comprising a plurality of electrode inputs, a plurality of amplifier channels, each amplifier channel comprising a differential input; and a programmable switch matrix configured to: 1) receive the plurality of electrode inputs, 2) route any electrode input to any differential input of the plurality of amplifier channels; and 3) enable reuse of at least one electrode input across two or more amplifier channels, such that the at least one electrode input is configured as either an active or a reference input for each of the two or more amplifier channels. Optionally, the programmable switch matrix comprises a plurality of multiplexers, and each multiplexer is configured to select from among the plurality of electrode inputs for each differential input. Optionally, the programmable switch matrix is configured to support mapping of 32 electrode inputs to at least 24 amplifier channels. Optionally, the programmable switch matrix includes buffer amplifiers configured to isolate the electrode inputs from capacitive loading.

Further disclosed herein is a method of forming a software-based montage in an intraoperative neurophysiological monitoring (IONM) system, the method comprising: acquiring a plurality of analog signals from a plurality of amplifier channels, each amplifier channel having a hardware-defined differential input, converting the analog signals to digital signals, and generating at least one virtual channel by computing a mathematical difference between two or more of the digital signals, wherein the virtual channel is not directly derived from a unique hardware-defined input pair. Optionally, generating the at least one virtual channel comprises subtracting a first digital signal from a second digital signal to create a derived signal. Optionally, an independently selected digital filter is applied to the virtual channel. Optionally, the virtual channel is stored and displayed alongside hardware-defined channels in a graphical user interface.

Further disclosed herein is an amplifier module for an intraoperative neurophysiological monitoring (IONM) system, comprising a plurality of electrode inputs, a programmable switch matrix configured to dynamically map the electrode inputs to a plurality of amplifier channels, wherein at least one electrode input is reused across two or more amplifier channels, a digitizer configured to convert analog signals from each amplifier channel to digital signals, and a signal processor configured to generate at least one virtual channel by combining the digital signals from two or more amplifier channels. Optionally, the signal processor generates the virtual channel by computing a time-aligned subtraction of two amplifier channel signals. Optionally, each amplifier channel is associated with independently configurable filter and gain settings. Optionally, the programmable switch matrix comprises a plurality of multiplexers and buffer amplifiers, and the signal processor is implemented within a logic module executing on a microcontroller or FPGA.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a schematic diagram of a clinical setup including a patient connected to a multi-modality intraoperative neurophysiological monitoring (IONM) system, in accordance with some embodiments of the present specification;

FIG. 2A is a circuit topology block diagram of the IONM system, in accordance with some embodiments of the present specification;

FIG. 2B is a front plan, exploded view of various components of the IONM system, in accordance with some embodiments of the present specification;

FIG. 3A illustrates a clinical setup of a portion of the IONM system with at least one multi-channel amplifier, in accordance with some embodiments of the present specification;

FIG. 3B is a switch matrix diagram illustrating at least one amplifier with an integrated programmable switch matrix module, in accordance with some embodiments of the present specification;

FIG. 3C is a diagram showing the relationship of a subset of amplifier channels generated from three electrode inputs using a programmable switch matrix module, in accordance with some embodiments of the present specification;

FIG. 3D is an illustration of an interface with a setup of electrode inputs for performing SSEP, MEP and EEG, in accordance with some embodiments of the present specification;

FIG. 3E is an illustration of a conventional interface with a setup of electrode inputs for performing SSEP, MEP and EEG;

FIG. 3F is an illustration of an interface with a setup of channel montages for performing SSEP, MEP and EEG, in accordance with some embodiments of the present specification;

FIG. 4A is a block diagram showing an AC-coupled programmable switch matrix module of an amplifier, in accordance with some embodiments of the present specification;

FIG. 4B is a block diagram showing a digital control loop circuit, implemented within a programmable switch matrix module of an amplifier, in accordance with some embodiments of the present specification;

FIG. 4C is a flowchart detailing a plurality of steps of a method of automatically adjusting offset voltage potential signal in an amplifier channel, in accordance with some embodiments of the present specification;

FIG. 5A is a block diagram showing a field programmable gate array (FPGA), in accordance with some embodiments of the present specification; and

FIG. 5B is a flowchart detailing a plurality of steps of a method of using the FPGA, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The terms “matrix” or “switch matrix” used in this disclosure may refer to a signal routing device which supports connecting any input to any output including multiple inputs to multiple outputs.

The term “module” or “engine” used in this disclosure may refer to computer logic utilized to provide a desired functionality, service or operation by programming or controlling a general purpose processor. Stated differently, in some embodiments, a module or engine implements a plurality of instructions or programmatic code to cause a general purpose processor to perform one or more functions. In various embodiments, a module or engine can be implemented in hardware, firmware, software or any combination thereof. The module or engine may be interchangeably used with unit, logic, logical block, component, or circuit, for example. The module or engine may be the minimum unit, or part thereof, which performs one or more particular functions. It should be noted herein that each hardware component is configured to perform or implement the plurality of instructions or programmatic code to which it is associated, but not limited to such functions.

It should be appreciated that embodiments of the present invention dramatically improve on the prior art, at least in part, as follows:

The present specification discloses, in embodiments, FPGA-Based Automatic Configuration Reset with Error Detection. A low-noise mechanism using an FPGA with two output registers and a comparator to detect corruption in multiplexer control lines and automatically reset them without human intervention. This employs dual output registers (one isolated from external pins) to verify configuration integrity via a comparator and generate an error flag, prompting automatic re-write of control lines via serial input from a microcontroller. This avoids clock noise and operator errors during surgical procedures and enables fault-tolerant, real-time reconfiguration.

The present specification also discloses, in optional embodiments, a Digital Control Loop for Real-Time DC Offset Correction. A digitally controlled DC-coupled loop with ADC/DAC and logic that applies a counter-based thresholding mechanism to nullify slow-varying electrode offset voltages. The loop uses a weighted counter with modulus logic to adjust DAC voltage only when needed, avoiding overcorrection. As a result, it reduces recovery time after electrical shocks (e.g., electrocautery), allowing better real-time monitoring.

The present specification also discloses, in embodiments, Expanded Channel Multiplexing Capability. 32 electrode inputs are dynamically mapped to up to 24 amplifier channels using a programmable matrix, with reuse of single electrodes across multiple differential channels. Traditional systems use 2:1 mapping (32 inputs→16 channels). This system uses input reuse and software-defined montaging to create more channels with fewer physical amplifiers. As a result, it reduces the hardware footprint, increases specificity of EEG/MEP/SSEP signals, and lowers cost.

### Multi-Modality IONM System

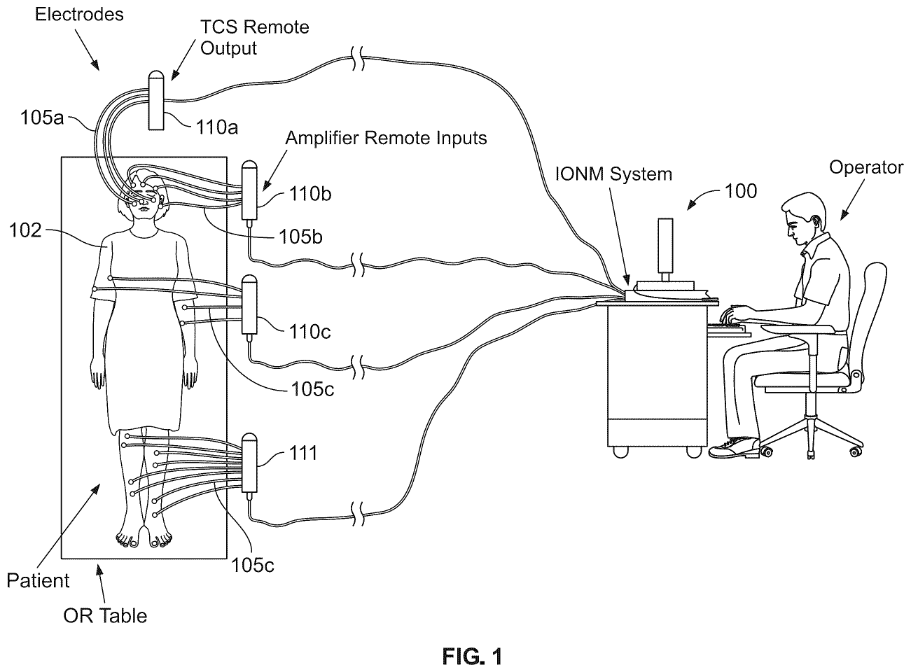

FIG. 1 is a clinical setup showing a patient connected to a multi-modality intraoperative neurophysiological monitoring (IONM) system 100, in accordance with some embodiments of the present specification. As shown, a plurality of stimulation and sensing/recording components are coupled to one or more regions of the patient's body 102. For example, a first set of components 105a may be stimulation electrodes deployed on the patient's scalp or placed directly over sections of the brain, in the form of a strip or grid electrode, monopolar probe, and/or bipolar probe. A second set of components 105b may be sensing/recording electrodes deployed on the patient's scalp or placed directly over sections of the brain, in the form of a strip or grid electrode, or depth electrodes that are positioned in deeper areas of the brain. A third set of components 105c may be sensing and/or recording electrodes positioned within a plurality of muscle sites or locations of the patient's body. In some embodiments, electrodes are small metal contacts typically made of stainless steel, platinum, tin, gold or silver covered with a silver chloride coating. In some embodiments, stimulation electrodes may include probes such as, but not limited to, a monopolar probe, a bipolar probe and/or a smart probe.

In some embodiments, using respective cables, each of the first set of components 105a is electrically coupled to a transcranial stimulator (TCS) remote output extender pod 110a, each of the second set of components 105b is electrically coupled to a first amplifier remote input extender pod 110b, each of a first subset of the third set of components 105c is electrically coupled to a second amplifier remote input extender pod 110c and each of a second subset of the third set of components 105c is electrically coupled to a third amplifier remote input extender pod 111. The remote extender pods 110a, 110b, 110c and 111 are, in turn, electrically coupled to the IONM system 100 via respective cables.

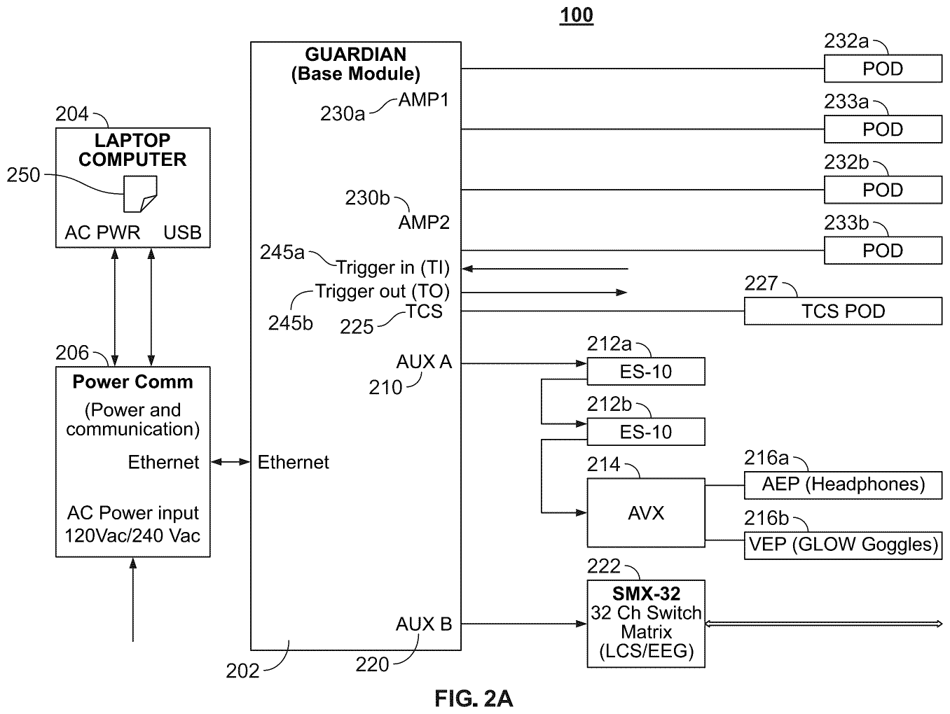

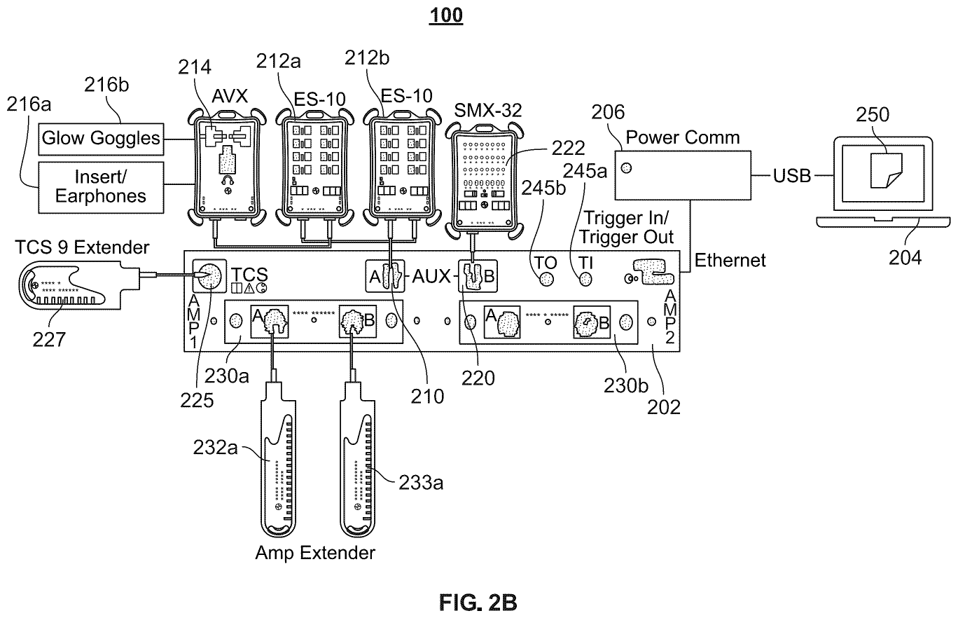

FIG. 2A is a circuit topology block diagram of the IONM system and FIG. 2B is a front plan, exploded view of various components of the IONM system, in accordance with some embodiments of the present specification. Referring now to FIGS. 2A and 2B, the IONM system 100 includes a base module 202 that is electrically coupled to a computing device 204, which, in embodiments, is configured as a controller, through a power module 206. The base module 202 includes a first port 210 for supporting electrical communication with an auditory and visual stimulator (AVX) module 214, which in turn, is in electrical communication with a first electrical stimulator 212a. Optionally, the first electrical stimulator 212a is in electrical communication with an optional second electrical stimulator 212b. The AVX module 214 is electrically coupled to an auditory evoked potential (AEP) stimulating device 216a (such as, for example, a headphone) and a visual evoked potential (VEP) stimulating device 216b (such as, for example, a pair of glow goggles).

The base module 202 includes a second port 220 for supporting electrical communication and coupling with a multi-channel EEG amplifier module 222. Further, base module 202 includes a TCS port 225 that includes a plurality of multiplexed outputs for supporting electrical coupling and communication with a TCS remote output extender pod 227. Base module 202 further includes a first multi-channel amplifier 230a and an optional second multi-channel amplifier 230b. The first multi-channel amplifier 230a is electrically coupled and in electrical communication with a first amplifier remote input extender pod 232a and an optional second amplifier remote input extender pod 233a. The second multi-channel amplifier 230b is electrically coupled to and in electrical communication with a third amplifier remote input extender pod 232b and an optional fourth amplifier remote input extender pod 233b.

The base module 202 also includes a trigger input port 245a and a trigger output port 245b. The trigger output port 245b is configured to produce an output signal or pulse that is time synchronized with the start of the amplifier recording collection of data. The trigger output port 245b is used to trigger an external stimulating device. The trigger input port 245a is configured for an external stimulating device to trigger the IONM device to start the amplifier recording collection of data.

In embodiments, the computing device 204 is configured to implement or execute an IONM software engine or module 250 (hereinafter referred to as an ‘IONM module’). In various embodiments, the IONM module 250 is configured to implement a plurality of instructions or programmatic code to: deliver a plurality of stimulation protocols or schedules (to one or more nerve structures of a patient) using one or more stimulation components (such as, for example, stimulation electrodes), generate a plurality of graphical user interfaces (GUIs) rendered on one or more display screens (that are coupled to the computing device 204) to display a plurality of intraoperative neurophysiological data acquired and stored using one or more sensing/recording components (such as, for example, sensing/recording electrodes) and extract a plurality of parameters related thereto in order to enable user-interaction with the system 100.

Thus, the IONM module 250 is configured to send commands or instructions in order to control and operate various elements, components and modules of the IONM system 100 to generate auditory, visual and/or electrical stimulations as well as acquire, display and store a plurality of intraoperative neurophysiological data from peripheral sensory and motor nerves, muscles, and the central nervous system, generated either spontaneously or elicited by stimuli. In various embodiments, the IONM module 250 is configured to support at least the following functions individually or in combination while acquiring, displaying and storing a plurality of intraoperative neurophysiological data related thereto: somatosensory, auditory, and visual evoked potentials (SSEP (somatosensory evoked potential), BAEP (brainstem auditory evoked potential), VEP (visual evoked potential), MEP (motor evoked potential)), electroencephalography (EEG), electrocorticography (ECoG), spontaneous and triggered electromyography (EMG), transcranial motor evoked potentials (TcMEP), direct cortical stimulation (DCS), direct nerve stimulation (TEMG, DNS), nerve conduction studies (NCS), and Train of Four (TOF) analysis.

### Extender Pods

Due to the presence of fluids and the lack of physical space at or near the operating room (OR) bed, it is not desirable to position the IONM system 100 at the OR bed. The electrode patient connections, therefore, need to be extended by a range of 3 to 10 meters using an extension cable. The extension cable typically comprises one of two configurations: a remote extender pod with touch-proof connections connected to a captive cable or a remote extender pod with touch-proof connections connected to a detachable cable. In some embodiments, a detachable cable configuration is preferred because it is more convenient to detach a pod with multiple electrode patient connections installed using a cable than to remove these multiple electrode patient connections from a pod with a captive cable when a patient needs to be moved or rotated on the OR bed.

For the detachable cable configuration, it is desirable to not have to reroute the detached extension cable if the patient is disconnected from the IONM system, rotated, and then reconnected to the IONM system 100. In some embodiments, the amplifier module 222 and each of the remote extender pods 227, 232a, 233a, 232b, and 233b includes an ID chip and detection circuitry that enables the IONM module 250 to associate assigned electrode patient connections with the specific remote extender pod to which they are connected. In some embodiments, the ID chip is in a “pod” (groups touchproof connectors) which is at the patient end of a cable connected to the amplifier or extender pod instead of having the ID chip in an adapter located at the amplifier or extender pod end of the cable. Therefore, an amplifier or a remote extender pod can be plugged into any extension cable and the IONM system 100 will recognize the identity of the amplifier or pod and remap the electrode patient connections regardless of which physical connection to the equipment is used. This configuration is similar to those described in U.S. Pat. Nos. 10,238,467, 11,241,297, 11,273,004, and 11,950,972, each of which is herein incorporated by reference in its entirety, with the pod with ID chip at the patient end of the cable rather than at the amplifier end. Not having to move the cables saves valuable time in the OR procedure and it prevents the misidentification of a patient's electrode connections. In some embodiments, since the ID chip cannot be read without causing disturbance to the biopotential signals in the detachable cable, a special circuit is required. The special circuit is detailed in U.S. Pat. Nos. 10,238,467, 11,241,297, 11,273,004, and 11,950,972, each of which is herein incorporated by reference in its entirety. This allows the IONM system to detect the attachment or removal of a pod and read the ID chip once, rather than requiring repeated polling of the ID chip serial line.

### Multi-channel Amplifiers

In a traditional amplifier, 2 physical electrode inputs are required to form 1 channel (an active-reference pair of inputs where “active” (input 1) minus “reference” (input 2) results in channel A). Therefore, a traditional 32 input amplifier provides 16 channels of data. If additional channels of data are needed, additional amplifiers must be added in parallel.

In accordance with aspects of the present specification, the first multi-channel amplifier 230a and the second multi-channel amplifier 230b are configured to have 32 patient connected electrode inputs that are multiplexed to up to 24 amplifier channels, instead of the traditional 16. This is achieved by assigning the patient connected electrode inputs to more than one amplifier channel. The theoretical limit of the number of channels for each of the amplifiers 230a, 230b is 496. Traditionally, when 1 electrode input is used in an active/reference channel it is no longer available to be paired in another channel montage. However, each of the amplifiers 230a, 230b is configured to allow 1 input to be used across multiple active/reference channels or referential channel setups. That is, any electrode input can be paired or multiplexed with one or more active/reference inputs and reused multiple times across different channels (Channel 1=input1-input2/Channel 2=input3-input4/Channel 3=input1-input3/Channel 3=input1-input4, and so forth).

The amplifiers 230a, 230b are configured to expand recording capabilities by allowing the user to montage across more channels. Assigning the same input in various pairs across multiple montages provides additional data points that relay specific information unique to each pairing. In a traditional 2:1 methodology, the number of channels often require two inputs to obtain the level of data required for a case. In comparison, a single amplifier, of the present specification, is configured to provide 8 additional channels of specificity, using less hardware than traditional IONM systems. By increasing the number of channels from 16 to 18, the amplifier is capable of sensing EEG signals.

In embodiments, each of the amplifiers 230a, 230b is configured to be used for sensing multiple discrete types of neurodiagnostic signals, simultaneously, such as EEG, SEP, nerve action potentials, muscles, and so forth. In this mixed mode use type of amplifier, each channel has independently configured filter and gain settings appropriate for the clinical modality in use (for example, EEG filter 1-70 Hz, SSEP filter 30-750 Hz, EMG filter 30-3000 Hz).

Thus, for each of the amplifiers 230a, 230b, increasing the number of amplifier channels available in a greater than 2:1 ratio reduces equipment costs, reduces setup complexity, and improves patient comfort by reducing the number of electrode leads attached. Also, since each individual amplifier has a separate ground and are electrically isolated from each other, creating channels from inputs sourced by separate amplifiers results in noisy poor quality active reference pairs. Therefore, for the amplifiers 230a, 230b, with the same number of inputs, greater sensitivity and specificity can be achieved from a single amplifier than using multiple amplifiers.

### Programmable Switch Matrix Module

In embodiments, each of the amplifiers 230a, 230b includes integrated, therewith, a programmable switch matrix module. The switch matrix modules are connected to a plurality of electrodes (positioned on various sites of a patient's scalp and/or body) through, for example, the remote extender pods 232a, 233a, 232b, and 233b. The switch matrix modules are also in communication with the IONM module 250 within computing device 204. Under the control of the IONM module 250, the switch matrix modules are configured to connect one or more of the plurality of sensing/recording components or electrodes to the amplifiers 230a, 230b.

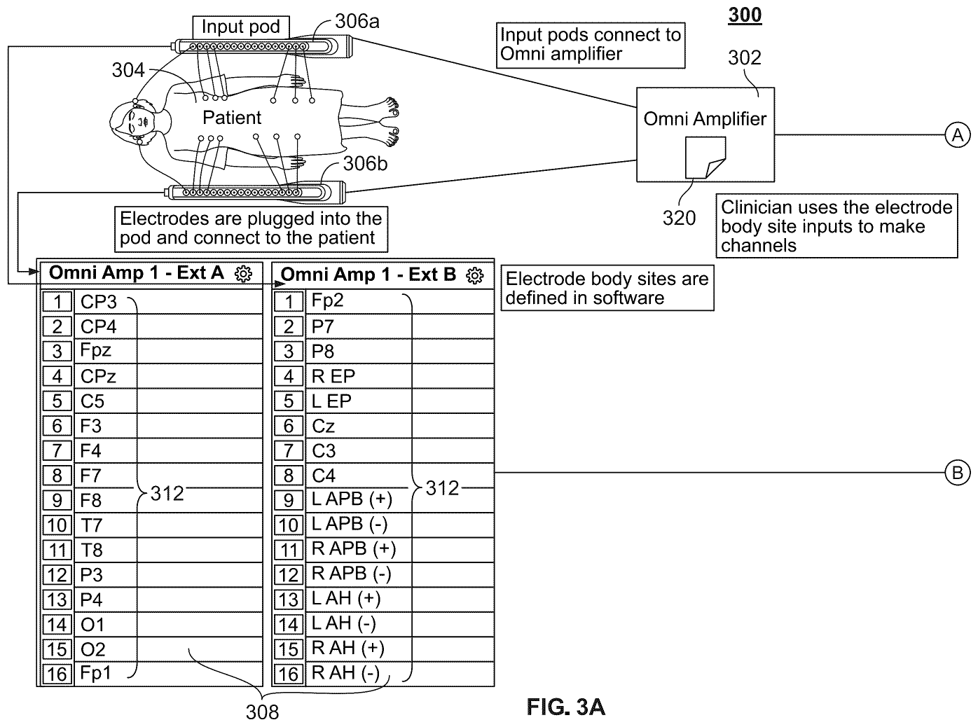

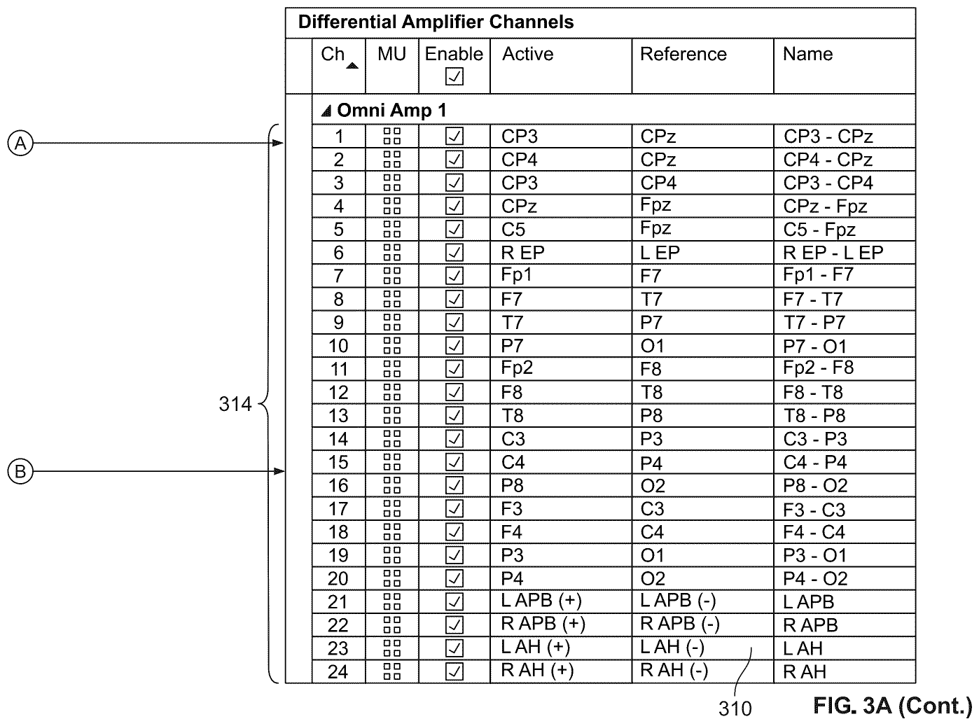

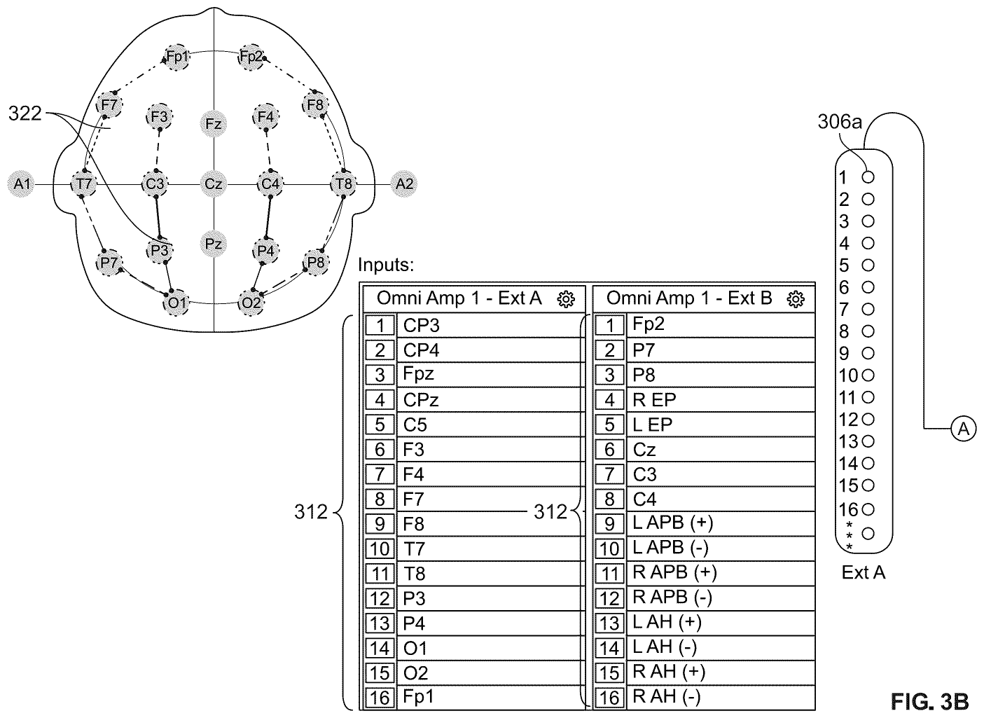

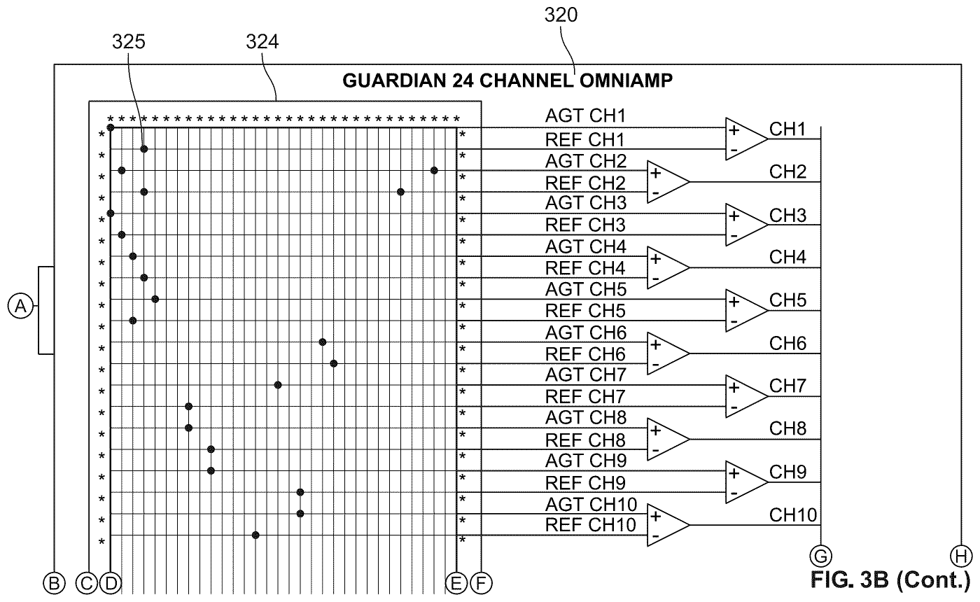

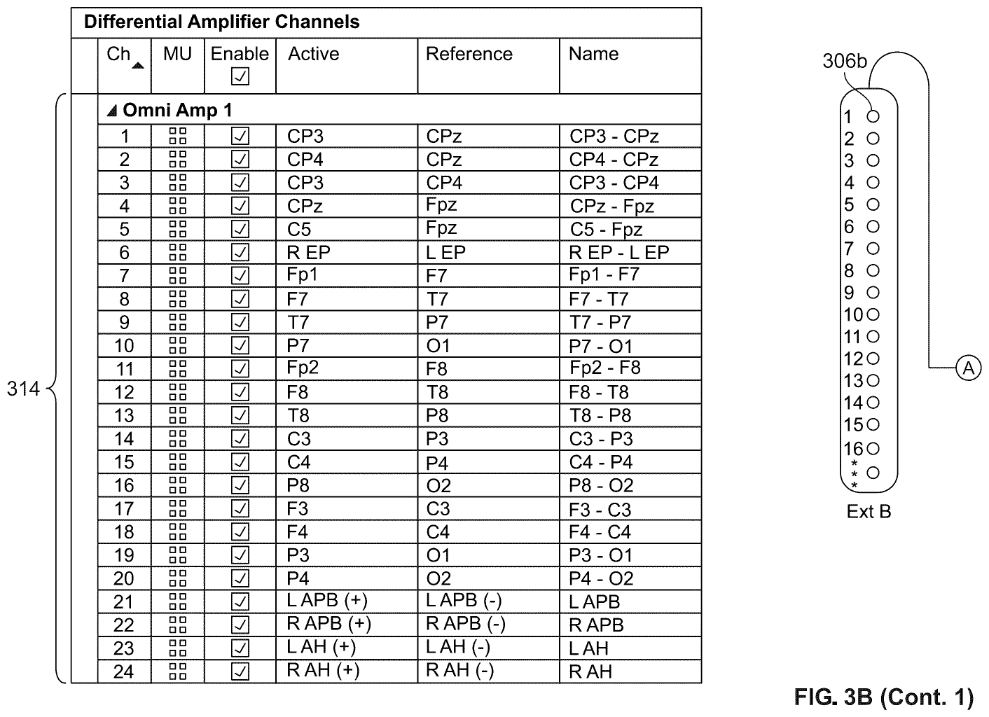

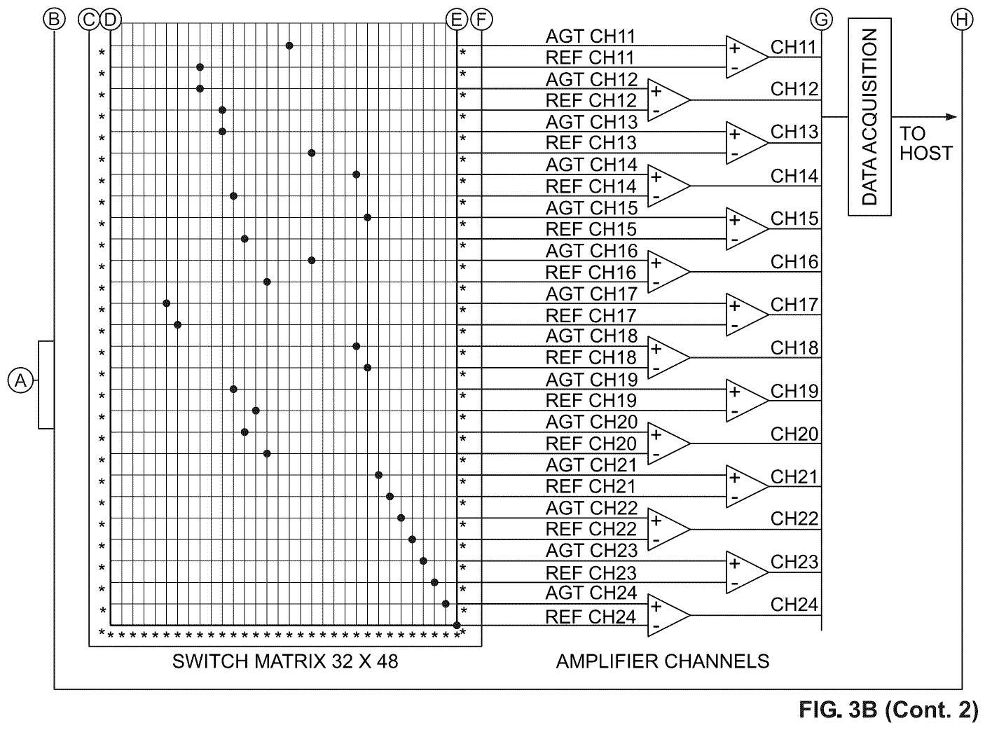

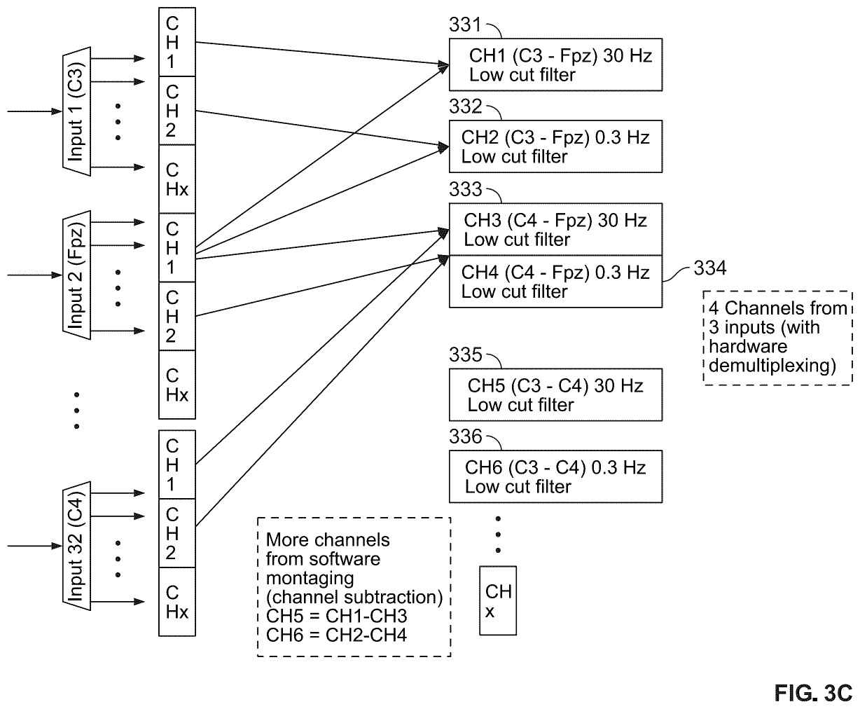

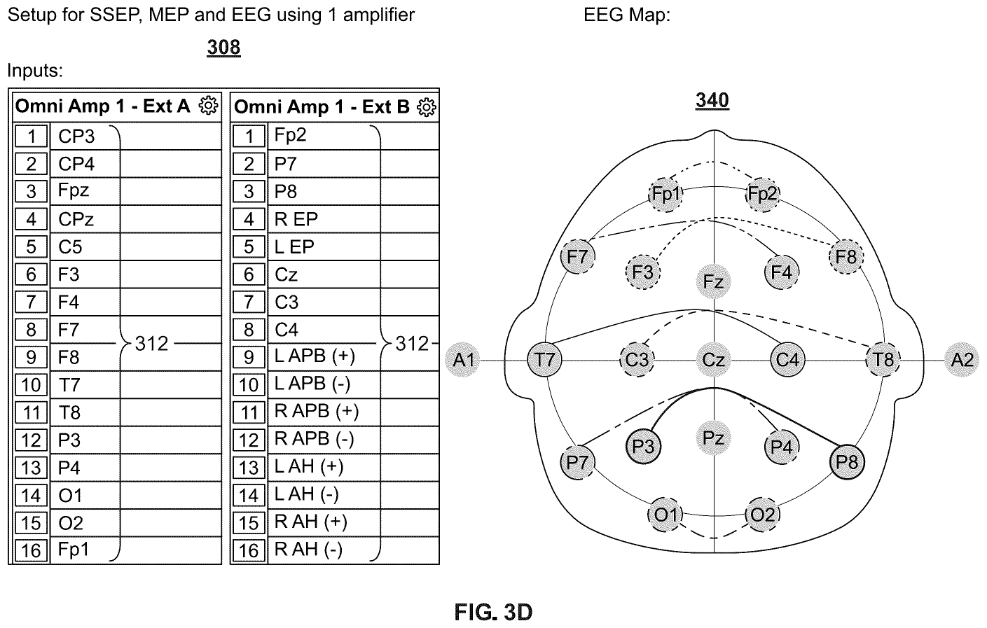

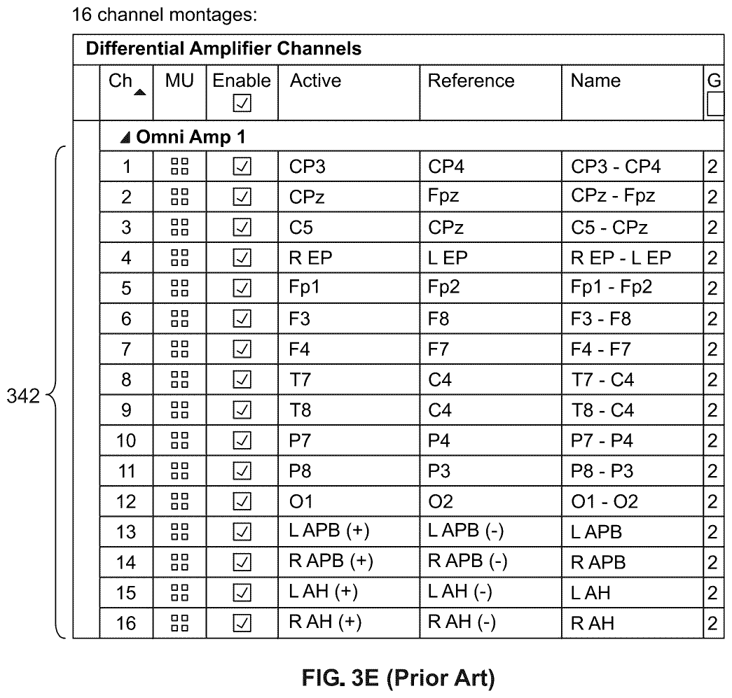

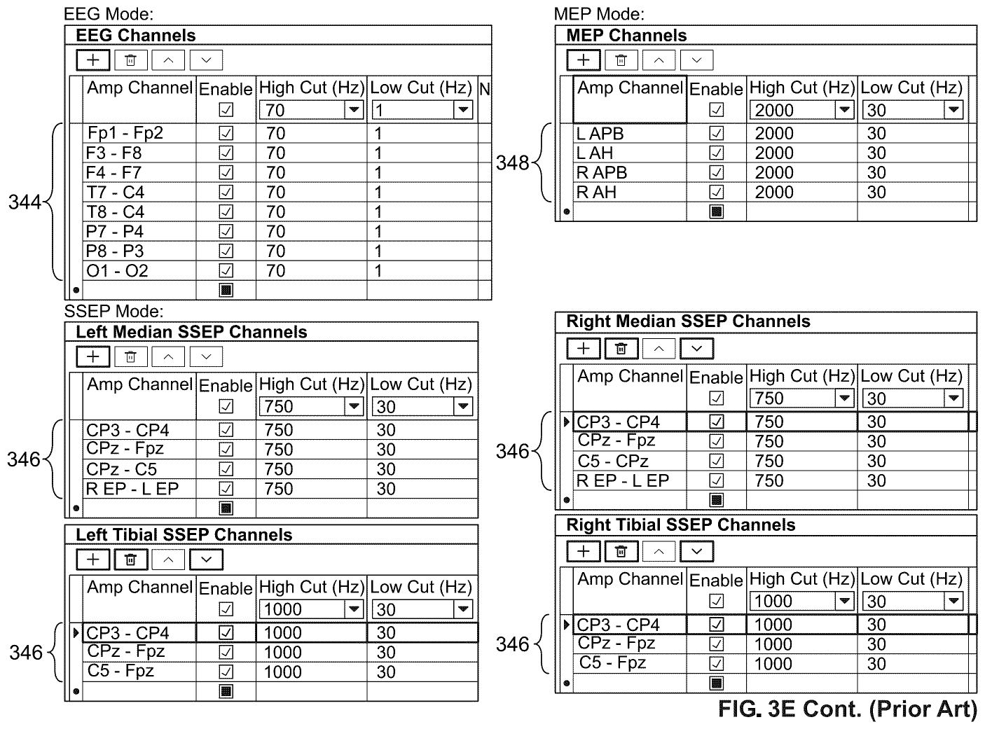

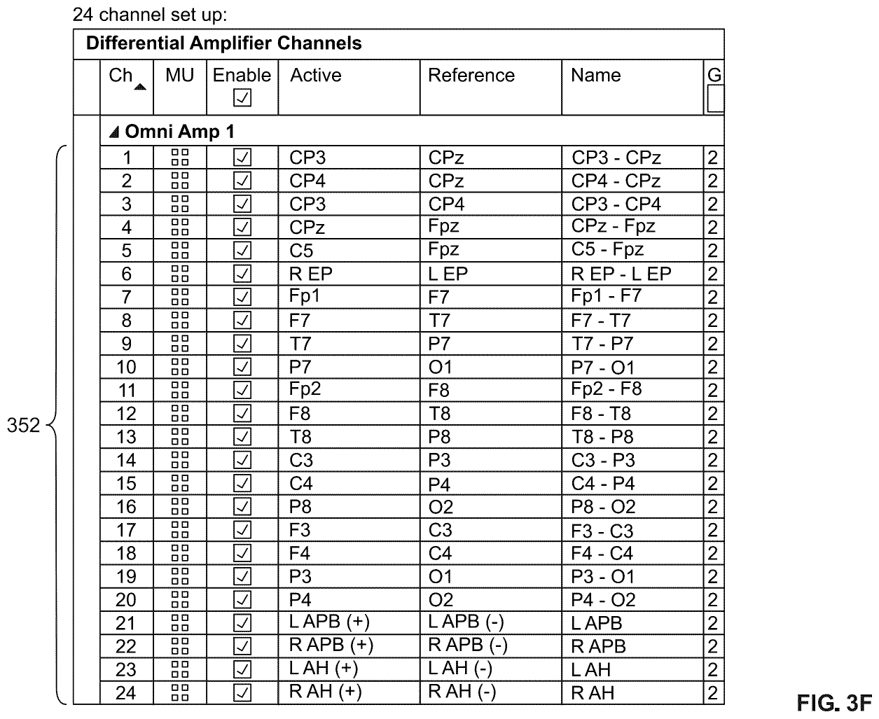

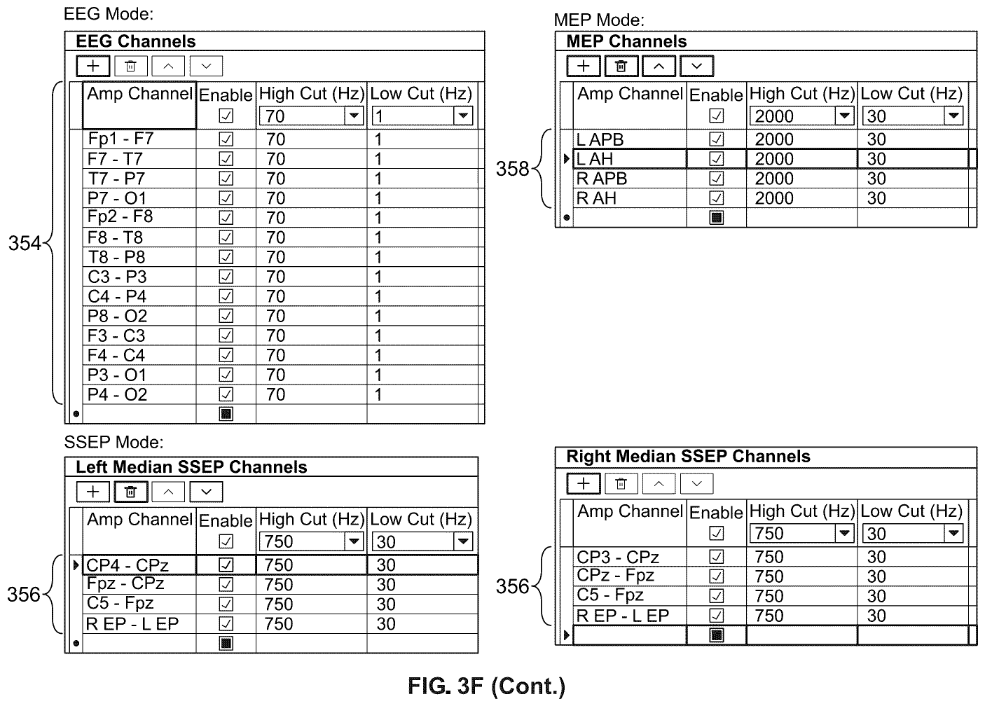

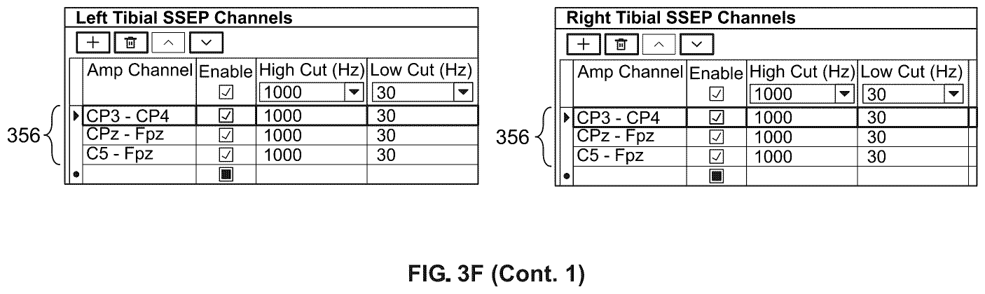

FIG. 3A illustrates a clinical configuration 300 of a portion of the IONM system 100 with at least one multi-channel amplifier 302, in accordance with some embodiments of the present specification. In embodiments, the at least one amplifier 302 is the same as one of the amplifiers 230a, 230b of FIGS. 2A and 2B. In a first configuration with a single amplifier 302, a clinician may place 32 electrodes on various body sites of a patient 304. The 32 electrodes connect to a first extender pod 306a and a second extender pod 306b (that is, 16 electrodes connect to the first extender pod 306a and another 16 electrodes connect to the second extender pod 306b) at the patient bedside. In the first configuration, the first extender pod 306a and the second extender pod 306b connect to a single amplifier 302. As shown, by way of example only, FIG. 3A is directed towards the first configuration that employs the single amplifier 302. The IONM module generates at least a first GUI 308 that defines the 32 electrode body sites 312. The IONM module further generates at least a second GUI 310 that enables the clinician to use inputs from the electrode body sites 312 to generate up to 24 channels 314. Since the at least one amplifier 302 is similar to the amplifiers 230a, 230b, it includes integrated, therewith, a programmable switch matrix module 320.

In a second configuration (such as described with reference to FIGS. 2A and 2B) with two amplifiers that are similar to the amplifier 302, a clinician places 64 electrodes on various body sites of the patient 304. The 64 electrodes connect to the first extender pod 306a and the second extender pod 306b as well as third and fourth extender pods (not shown) at the patient bedside. In this second configuration, the first extender pod 306a and the second extender pod 306b as well as the third and fourth extender pods connect to up to two amplifiers in total. In embodiments, the number of electrodes necessary for IONM monitoring and the configuration is based on the complexity of the surgery or procedure.

In both the single amplifier configuration and the two-amplifier configuration, the amplifiers are in communication with a computing device that is configured to execute an IONM module (such as, the IONM module 250 of FIGS. 2A and 2B).

FIG. 3B is a switch matrix diagram illustrating the at least one amplifier 302 with an integrated programmable switch matrix module 320, in accordance with some embodiments of the present specification. The switch matrix module 320 is shown connected to a plurality of electrodes 312 positioned on various body sites of the patient 304. In some embodiments, the plurality of electrodes is 32. The switch matrix module 320 includes circuit paths 324 that, in some embodiments, are arranged in 32 columns and 48 rows. In some embodiments, the programmable switch matrix module 320, in response to commands received from the IONM module, is operable to change a circuit path configuration to enable generation of up to 24 amplifier channels 314. The columns correspond to the 32 electrode connections at the first extender pod 306a and the second extender pod 306b. The 48 rows correspond to active and reference inputs of the 24 channels 314.

Any electrode connection (that is, any column) can be connected to any one or more active and reference inputs (that is, the rows). As shown in the switch matrix module 320, of FIG. 3B, a ‘dot’ 325 at the intersection of the row and column indicates an electrode connection to an active or reference channel input. Multiple ‘dots’ 325 on a column indicate one electrode connection to multiple active or reference channel inputs. Thus, in embodiments, there is no restriction to the number of active or reference channel inputs for a specific electrode connection.

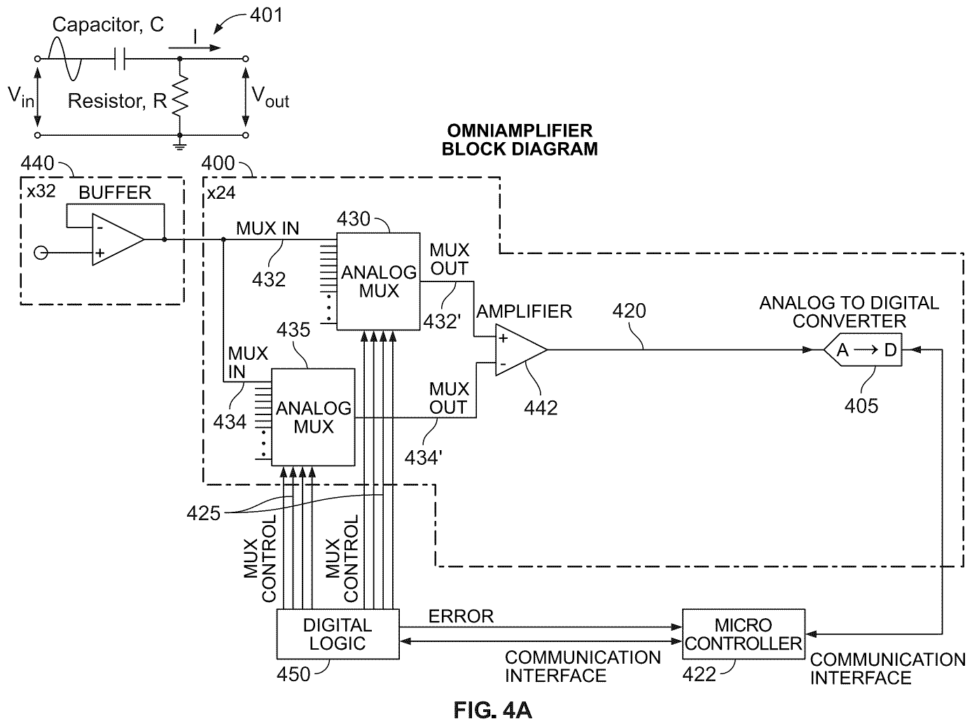

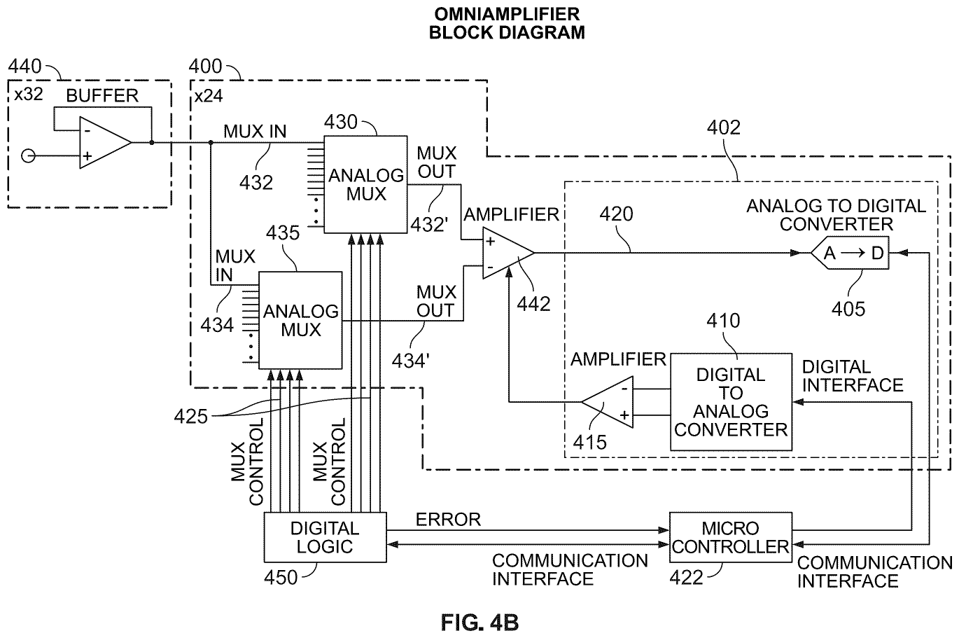

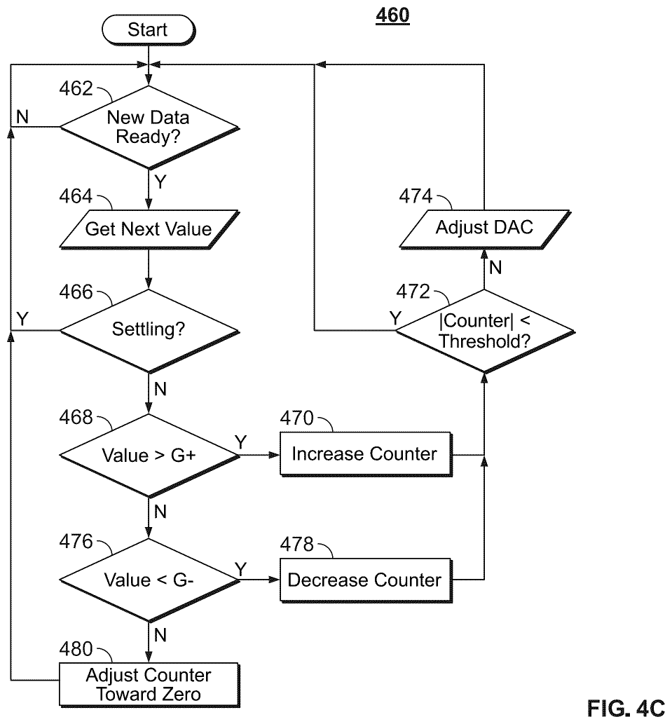

In some embodiments, each of the 24 amplifier channels consists of a differential active input and a reference input. A connection between any of the 32 inputs to any of the 24 active or 24 reference inputs of the 24 differential amplifier channels is required. To accomplish this, the input signal is connected to a low noise buffer amplifier (shown as element 440 in FIGS. 4A and 4B). The buffer amplifiers are configured as unity gain amplifiers so as not to introduce gain errors that will degrade the common-mode rejection of the amplifier 302. The buffer amplifier eliminates the capacitive loading on the input signals caused by direct connection to the multiplexers and differential amplifiers. The output of each buffer amplifier is connected to two 16:1 input multiplexers for the active input of the differential amplifier and to another two 16:1 input multiplexers for the reference input to the same differential amplifier. In total, the output of each buffer amplifier is connected to a total of 96 16:1 input multiplexers. It should be appreciated, that 16:1 refers to 16 input lines and 1 output line of a multiplexer.

FIG. 3C illustrates the relationship of a subset of amplifier channels (of the 24 channels) generated from three electrode inputs (of the 32 inputs) using the programmable switch matrix module 320, in accordance with some embodiments of the present specification. As shown, a first channel 331 is generated as a difference of C3 and Fpz inputs with a filter setting of 30 Hz, a second channel 332 is generated as a difference of C3 and Fpz inputs with a filter setting of 0.3 Hz, a third channel 333 is generated as a difference of C4 and Fpz inputs with a filter setting of 30 Hz, a fourth channel 334 is generated as a difference of C4 and Fpz inputs with a filter setting of 0.3 Hz. Thus, the switch matrix module 320 is configured to enable the formation of four channels 332, 332, 333 and 334 from three inputs C3, C4 and Fpz (in various combinations as above) as a result of hardware based multiplexing. Additional channels, such as a fifth channel 335, may be generated as a difference of the third channel 333 and the fourth channel 334 with a filter setting of 30 Hz, and a sixth channel 336 may be generated as a difference of the third channel 333 and the fourth channel 334 with a filter setting of 0.3 Hz. It should be appreciated that the additional channels are formed from software-based montaging enabled by the IONM module. Thus, each channel has independently configured filter and gain settings (that is, the filter and gain settings are by channel instead of by input) and any electrode input can be paired or multiplexed with one or more active/reference inputs and reused multiple times across different channels.

FIGS. 3A, 3B and 3D illustrate configurations of electrode inputs for performing SSEP, MEP and EEG, in accordance with some embodiments of the present specification. The IONM module generates the first GUI 308 for displaying the 32 electrode body sites 312. Also shown is an EEG electrode map 340 for the patient's scalp. As shown in the prior art GUIs of FIG. 3E, traditionally, the 32 electrode inputs are used to generate 16 channel montages 342 comprising 8 channel montages 344 of transverse non-bipolar EEG, 4 channel montages 346 of SSEP, and 4 channel montages 348 of MEP. This is the least specific (non-sensitive) set up for EEG, SSEP and MEP. Thus, in the 2:1 channel montaging methodology, less channels are available, wherein the fewer number of channels are also non-specific.

In contrast, as shown in the GUIs of FIG. 3F, the IONM system of the present specification uses the 32 electrode inputs to generate 24 channel montages 352 comprising 14 channel montages 354 of bipolar EEG, 4 channel montages 356 of SSEP specific to upper/lower, and 4 channel montages 358 of MEP. This is a more specific (sensitive) set up for EEG, SSEP and MEP compared to the traditional 2:1 channel montaging.

### Addressing DC-Offset Voltage Potential in Electrodes

Referring back to FIG. 2A, as is known, electrodes placed on a patient exhibit an offset voltage potential between any two electrodes. The offset voltage potential may range between 0.2 V and 1 V. The acceptable offset range is variable depending on the design of the amplifier. This electrode offset voltage potential may vary slowly over time. Stated differently, electrode DC levels drift over time. If adjustments are not made to account for the drift, the amplifier channel will saturate. The IONM amplifier, such as the amplifiers 230a, 230b, must amplify the microvolt bio-potential signal detected by the electrodes while rejecting the offset voltage potential.

### AC-Coupling of the Amplifier

In some embodiments, AC coupling is used to eliminate DC offset voltages from electrodes so that small but meaningful neural signals (which are AC in nature) can be accurately observed and amplified. FIG. 4A is a block diagram showing an AC-coupled programmable switch matrix module 400 of an amplifier, used to eliminate the offset voltage potential in electrodes that are in electrical communication with the amplifier and that are placed on a patient, in accordance with some embodiments of the present specification. The switch matrix module 400 illustrates, as a non-limiting example, a first multiplexer 430 configured to allow, under control commands from a logic module 450, a first electrode input 432 (from a plurality of electrode inputs) on its corresponding output line 432′. Similarly, a second multiplexer 435 is configured to allow, under control commands from the logic module 450, a second electrode input 434 (from the plurality of electrode inputs) on its corresponding output line 434′. An amplifier channel 420 (of a differential amplifier 442) is then configured as a difference of the first electrode input 432 and the second electrode input 434.

In some embodiments, a high pass filter 401 is positioned in series with electrode inputs of the amplifier. In an embodiment, the high pass filter 401 is an RC circuit where Vin is an incoming signal from an electrode, C is a coupling capacitor, R is an input resistor, and Vout is the voltage delivered to the amplifier input. AC coupling, via the capacitor C, eliminates DC offset voltages from the electrode inputs while allowing neural AC signals to pass through to the amplifier.

In some embodiments, the logic module 450 is a part of the switch matrix 400 and includes a plurality of instructions or programmatic code which when executed by a microprocessor 422 is configured to automatically reset the control lines 425 (for configuring the channel 420) in response to detecting that the control lines 425 are corrupted. In embodiments, the microcontroller 422 is in data communication with the IONM module 250 (of FIGS. 2A and 2B). In addition, a low noise buffer amplifier 440 configured as a unity gain amplifier is also included. The buffer amplifier 440 eliminates the capacitive loading on input signals caused by direct connection to the first multiplexer 430 and second multiplexer 435 and the differential amplifier 442.

The ADC 405 is configured to digitize the analog signal at the amplifier channel 420 (of a plurality of amplifier channels), wherein the digitized signal is then processed by the microcontroller 422 (based on execution of a plurality of instructions or programmatic code of the logic software module 450).

### Automatic DC Offset Adjustment

In some optional, alternative embodiments, the hardware circuitry of a control loop 402 within programmable switch matrix module of the amplifier, responsive to the commands of microcontroller 422, is configured to eliminate the offset voltage potential by a DC coupled amplifier.

FIG. 4B is a block diagram showing a digital control loop circuit 402, implemented within a programmable switch matrix module 400 of an amplifier, to compensate for the offset voltage potential in electrodes (that are in electrical communication with the amplifier) placed on a patient, in accordance with some embodiments of the present specification. As shown in FIG. 4B, the digital control loop 402 comprises an ADC (Analog to Digital Converter) 405, a DAC (Digital to Analog Converter) 410 and analog circuitry 415. The analog circuitry 415 is configured to scale and lowpass filter the signals from the DAC 410. As discussed earlier, the switch matrix module 400 illustrates, as a non-limiting example, the first multiplexer 430 configured, under control commands from the logic module 450, to allow the first electrode input 432 (from a plurality of electrode inputs) on its corresponding output line 432′. Similarly, the second multiplexer 435 is configured, under control commands from the logic module 450, to allow the second electrode input 434 (from the plurality of electrode inputs) on its corresponding output line 434′. The amplifier channel 420 (of a differential amplifier 442) is then configured as a difference of the first electrode input 432 and the second electrode input 434.

In some embodiments, the logic module 450 is a part of the switch matrix 400 and includes a plurality of instructions or programmatic code which when executed by a microprocessor 422 is configured to automatically reset the control lines 425 (for configuring the channel 420) in response to detecting that the control lines 425 are corrupted. In embodiments, the microcontroller 422 is in data communication with the IONM module 250 (of FIGS. 2A and 2B). Also included is the low noise buffer amplifier 440 configured as a unity gain amplifier. The buffer amplifier 440 eliminates the capacitive loading on input signals caused by direct connection to the first multiplexers 430 and second multiplexer 435 and the differential amplifier 442.

The ADC 405 is configured to digitize the analog signal at the amplifier channel 420 (of a plurality of amplifier channels) containing the offset voltage potential, wherein the digitized signal is then processed by the microcontroller 422 (based on execution of a plurality of instructions or programmatic code of the logic software module 450) to detect the offset voltage potential. In response to the detected offset voltage potential, the microcontroller 422 is configured to set the DAC 410 potential to a value (of counteracting voltage potential that is equal and opposite to the detected offset voltage potential) that nulls out the offset voltage through the analog circuitry 415. Stated differently, the microcontroller 422 is configured to detect and track the DC offset for individual channels, such as, for example, channel 420.

FIG. 4C is a flowchart detailing a plurality of steps of a method 460 of automatically adjusting an offset voltage potential signal in an amplifier channel, in accordance with some embodiments of the present specification. Referring to FIGS. 4B and 4C, in embodiments, the logic module 450 includes a plurality of instructions or programmatic code which when executed cause the microcontroller 422 to perform the plurality of steps of the method 460.

In embodiments, method 460 includes maintaining a counter that represents how long the signal data (indicative of offset voltage potential), at the amplifier channel 420, has exceeded an offset guard threshold. In some embodiments, a non-limiting example value of the offset guard threshold is 700. The counter is “weighted” to count in larger steps when the threshold is exceeded and counts slower when in range. Method 460 is therefore biased towards making quick adjustments and letting the signal settle down more slowly. A counter value of 0 indicates that the signal has been in range for at least 200 milliseconds, in a typical configuration. The counter is increased to be positive if the signal has exceeded a positive guard limit, and the counter is decreased to be negative if the signal has exceeded a negative guard limit. The positive and negative guard limits are specified as a percentage of the available range. The guard limits are thresholds as a percentage of the full-scale range. For example, in some embodiments, the full-scale range of a signed 16-bit value is −32768 to +32767, resulting in guard limits, set at 70%, of −22937 and +22936. Thus a series of oscillations will effectively cancel out and will result in no offset adjustment since the net gain is 0.

However, when the counter exceeds the counter threshold and it is determined that an adjustment is needed, a voltage value of the DAC 410 is adjusted based on the last signal value received that exceeded the threshold. That is, the DAC voltage on the channel 420 will change by the offset voltage detected in the signal data in an attempt to counteract the effect of the DC offset. A timestamp of when the adjustment occurred as well as the voltage delta of the DAC 410 adjustment is sent to the IONM module 250. The IONM module 250 is configured to adjust the digital filters being applied to the signal data at the specified time to compensate for the sudden DC shift in signal due to the DAC 410 adjustment. After the DAC 410 adjustment is made, a settling period is observed where no additional adjustments are allowed until the settling period expires.

Referring now to FIGS. 4B and 4C, at step 462, the microcontroller 422 is configured to determine if new signal data indicative of an offset voltage potential is available from the ADC 405 that digitizes the analog signal, containing the offset voltage potential, at the amplifier channel 420. If no new signal data is detected, then the amplifier channel 420 is continued to be polled in order to detect new signal data. If new signal data is detected, then, at step 464, a first value indicative of the offset voltage potential (in the new signal data) is extracted or acquired.

At step 466, it is determined if the predefined, yet customizable, settling period is not over. In various embodiments, the settling period or practical minimum time required is determined by the filter circuitry in 415. That is, any adjustment will not reach its intended value until the filter in 415 settles. Microcontroller 422 uses its internal system time to determine when the period is over by comparing the current time with the time at which the adjustment began. If the settling period is not over, then no additional adjustments are allowed and the flow moves back to step 462 where the amplifier channel 420 is continued to be polled in order to detect new signal data.

If the settling period is over, then, at step 468, it is determined if the first value indicative of the offset voltage potential exceeds the predefined, yet customizable, positive guard limit. If yes, then the flow moves to step 470 where the counter is continued to be increased (in a positive direction) by incremental value steps, until the first value indicative of the offset voltage potential does not exceed the positive guard limit. In embodiments, the counter increases by 2 every time the guard limit is exceeded and is incremented as long as the guard limit is exceeded. Next, at step 472, it is determined if a modulus of the incremented counter value does not exceed a predefined, yet customizable, counter threshold. If the modulus of the incremented counter value does not exceed the counter threshold, then no offset adjustments are implemented and the flow moves back to step 462 where the amplifier channel 420 is continued to be polled in order to detect new signal data. If the modulus of the incremented counter value exceeds the counter threshold, then, at step 474, a voltage value of the DAC 410 is adjusted based on the first value indicative of the offset voltage potential. That is, the voltage value of the DAC 410 is adjusted to be equal to, but opposite in direction to, the first value indicative of the offset voltage potential. The voltage value of the DAC 410 is applied to the amplifier channel 420 in order to counteract the effect of the first value indicative of the offset voltage potential (in the new signal data). Thereafter, the flow moves back to step 462.

However, if at step 468, it is determined that the first value indicative of the offset voltage potential does not exceed the positive guard limit then the flow moves to step 476. At step 476, it is determined if the first value indicative of the offset voltage potential exceeds the predefined, yet customizable, negative guard limit. If yes, then the flow moves to step 478 where the counter is continued to be decreased (in a negative direction) by decremented value steps, until the first value indicative of the offset voltage potential does not exceed the negative guard limit. The counter is decremented as long as the guard limit is exceeded. Thereafter the flow moves to step 472, where it is determined if a modulus of the decremented counter value does not exceed the predefined, yet customizable, counter threshold. If the modulus of the decremented counter value does not exceed the counter threshold, then no offset adjustments are implemented and the flow moves back to step 462 where the amplifier channel 420 is continued to be polled in order to detect new signal data. If the modulus of the decremented counter value exceeds the counter threshold, then the flow moves to step 474, where a voltage value of the DAC 410 is adjusted based on the first value indicative of the offset voltage potential. That is, the voltage value of the DAC 410 is adjusted to be equal to, but opposite in direction to, the first value indicative of the offset voltage potential. The voltage value of the DAC 410 is applied to the amplifier channel 420 in order to counteract the effect of the first value indicative of the offset voltage potential (in the new signal data). Thereafter, the flow moves back to step 462.

However, if at step 476, it is determined that the first value indicative of the offset voltage potential does not exceed the negative guard limit then the flow moves to step 480 where the value of the counter is adjusted towards zero in single count increments or decrements. Thereafter, the flow moves back to step 462.

### Automatic Reset in Response to Detection of Error State

Referring again to FIGS. 4A and 4B, control lines 425 are configured to set the configuration of channel 420, which can include electrode input multiplexing, channel amplifier gain, and channel filter frequency. If the control lines 425 fail to set the configuration of a channel correctly, this can result in improper monitoring of the patient biopotential signals. Since external electrical interference may disturb the control lines 425, standard design topologies of using latching multiplexers, such as cross-point switches, or discrete digital latches do not indicate if the latches are disturbed. A direct connection of the control lines 425 to the microcontroller 422 is also not desirable since the microcontroller 422 may add electrical noise to the control lines 425 and cause noise to be added to the biopotential signal being switched by the multiplexers 430 and 435 (that are 16:1 multiplexers, in accordance with some embodiments).

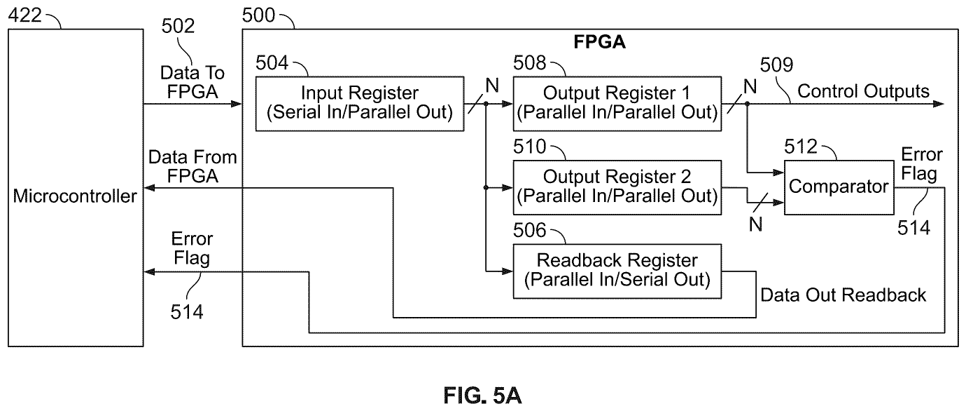

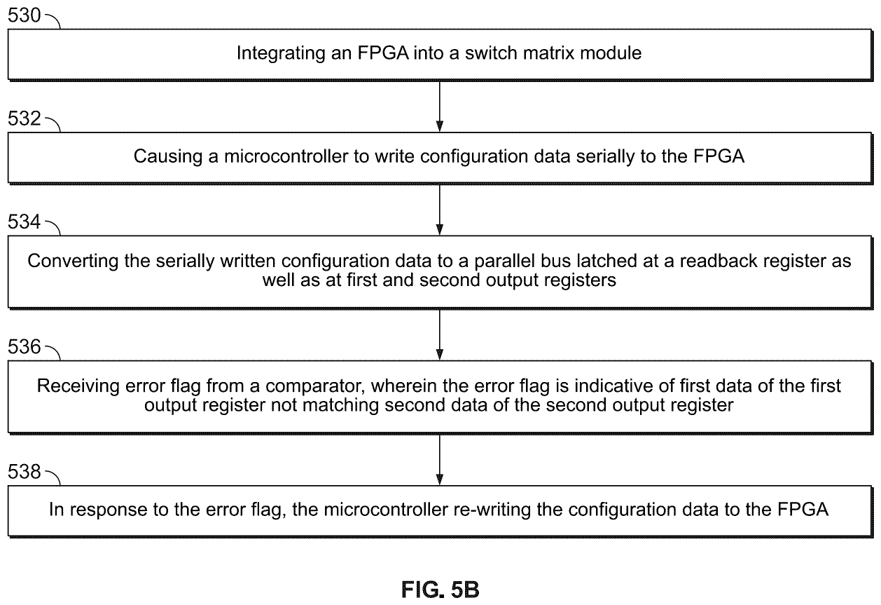

FIG. 5A shows a field programmable gate array (FPGA) 500 configured to automatically reset the control lines 425, in accordance with some embodiments of the present specification. In embodiments, the FPGA 500 is implemented within the programmable switch matrix module 400. Referring now to FIGS. 4A, 4B and 5, the logic module 450 is configured to implement a low noise method, by the FPGA 500, to a) detect when the control lines 425 have been corrupted and b) in response, automatically reset the control lines 425 and notify microcontroller 422 by setting an error signal 514. That is, the logic module 450 includes a plurality of instructions or programmatic code which when executed cause the microcontroller 422 to write a serial data stream 502 to the FPGA 500 to set its internal latches in response to detecting corruption of the control lines 425.

Thus, the logic module 450 is configured to monitor the state of the internal latches of the FPGA 500. If the monitored state of the internal latches is indicative of being disturbed or corrupted (such as, by external electrical interference), the logic module 450 automatically generates an error flag. The error flag is read by the microcontroller 422 which, in response, re-writes the configuration data (including input multiplexing, channel amplifier gain, and channel filter frequency) of the amplifier channel 420 to set the internal latches of the FPGA 500 correctly.

In embodiments, amplifier channel configuration consists of at least one of input selection, gain, and low-cut filter. Each channel has configurable data indicative of two inputs: an active input and a reference input. For all channels, the active input can be any of 1 to 32 inputs, for example. The reference input can be any of 1 to 32 inputs, for example. Each channel further has configurable data indicative of a channel amplifier gain. Gain is a ratio of an output signal to an input signal. An example set of gain settings is 200X, 400X, 800X, 2500X, 5000X, and 10000X. Still further, each channel has configurable data indicative of a frequency of a low-cut filter. A low-cut filter attenuates or reduces frequencies below a specific cutoff point while allowing frequencies above that point to pass through. An example set of low-cut settings is 0.1 Hz, 3 Hz, or 10 Hz.

It should be appreciated, that in the present embodiment, the error flag mechanism does not use a continuously running clock. The presence of a continuously running clock in the FPGA 500 will cause noise on the multiplexer control lines 425 and onto the biopotential signals running through the analog multiplexers 430, 435. It should also be appreciated, that unlike the current state of the art, operator intervention is not required to detect that the patient biopotential input signals are not configured correctly and to take corrective action such as pressing a hardware or software reset button.

One source of external electrical interference is an Electro-Surgical Unit (ESU), used to cut or cauterize tissue in the operating room environment. To prevent excessive rewriting of the latches during use of the ESU, the amplifier incorporates a high frequency noise detector that is monitored by the microcontroller 422. In embodiments, the high frequency noise detector is similar to the detectors disclosed in U.S. Pat. No. 11,596,337 and United States Patent Application Publication Number 2023-0255534, both of which are herein incorporated by reference in their entirety. The microcontroller 422 suspends writing channel configuration to the FPGA 500 when the high frequency noise detector indicates noise is present and will only rewrite the FPGA latches when the noise is gone for a predefined period of time and an error flag indicates that a reset is needed. Biopotential signals are not actively monitored when external high frequency noise is present because the noise is visible on the signals, obscuring the data. Therefore, waiting until the noise source is gone to reset the FPGA latches does not affect the use of the system.

In embodiments, the microcontroller 422 writes the configuration data 502 serially to the FPGA 500. The logic module 450 is configured to convert the serially written configuration data 502, through an input register 504, to a parallel bus which is latched at one readback register 506 and two output registers-first output register 508 and second output register 510. The serial to parallel register 504 comprises a storage element that is configured to take a single input line (serial data) and make all the outputs available in a parallel format. In embodiments of the present specification, the FPGA provides a plurality of output control lines that are written using a single input data line from the microcontroller, which reduces the number of pins used on the microcontroller. The input register 504 provides the flexibility to defer updating the output and readback registers until it is deemed appropriate to do so. For example, all data can be written serially to the input register 504, and then all the outputs can be updated at once at the end of the sequence (as opposed to the outputs continuously updating while the input sequence is being written). The readback register 506 provides a mechanism to read back the configuration data serially by the microcontroller 422. The first output register 508 has its outputs 509 connected to external pins used as control lines 425 to the multiplexers 430, 435. The second output register 510 is not connected to any external pins. First data of the first output register 508 and second data of the second output register 510, each containing configuration data, are provided to a comparator 512 which will assert the error flag 514 back to the microcontroller 422 in the case that the first data of the first output register 508 and second data of the second output register 510 do not match.

The error flag 514 is read by the microcontroller 422 which, in response, re-writes the configuration data to the FPGA 500.

FIG. 5B is a flowchart detailing a plurality of steps of a method of using the FPGA 500 to detect configuration error(s) in and automatically reset a plurality of control lines of one or more multiplexers of a switch matrix module, in accordance with some embodiments of the present specification. Referring now to FIGS. 5A and 5B, at step 530, the FPGA 500 is integrated into the switch matrix module such that the FPGA 500 is in data communication with the microcontroller 422 and the plurality of control lines to the one or more multiplexers, wherein the plurality of control lines configures each channel of a plurality of channels of an amplifier.

At step 532, based on instructions received from logic module 450 (FIG. 4A), the microcontroller 422 writes configuration data serially to the FPGA 500. At step 534, the serially written configuration data is converted to a parallel bus latched at the readback register 506 as well as at first output register 508 and second output register 510, respectively.

At step 536, an error flag is received from the comparator 512, wherein the error flag is indicative of first data of the first output register 508 not matching second data of the second output register 510. At step 538, in response to the error flag, the microcontroller 422 re-writes the configuration data to the FPGA 500. In embodiments, the configuration data is indicative of electrode input multiplexing, channel amplifier gain, and channel filter frequency.

The above examples are merely illustrative of the many applications of the systems and methods of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. An electronic circuit adapted to operate as a switch matrix in an amplifier of an intraoperative neurophysiological monitoring (IONM) system, wherein the amplifier comprises a plurality of channels, the electronic circuit comprising:
a microcontroller;
a plurality of control lines in electrical communication with one or more multiplexers, wherein the plurality of control lines is adapted to configure each channel of a plurality of channels of the amplifier;
a field programmable gate array (FPGA) in data communication with the microcontroller and the plurality of control lines, wherein the FPGA comprises an input register, a parallel bus latched at a readback register, at a first output register and at a second output register, and a comparator, wherein the comparator is configured to receive first data of the first output register and second data of the second output register; and
a logic module comprising a plurality of programmatic instructions that, when executed, cause the microcontroller to:
write configuration data serially to the FPGA;
receive an error flag from the comparator, wherein the error flag is indicative of the first data not matching the second data; and
re-write, in response to the error flag, the configuration data to the FPGA.

2. The electronic circuit of claim 1, wherein the logic module further comprises a plurality of programmatic instructions that, when executed, cause the microcontroller to convert serially written configuration data to the parallel bus.

3. The electronic circuit of claim 1, wherein the readback register is configured to allow readback of the configuration data serially by the microcontroller.

4. The electronic circuit of claim 1, wherein the first output register is connected to external pins in electrical communication with the plurality of control lines.

5. The electronic circuit of claim 4, wherein the second output register is not connected to any external pins.

6. The electronic circuit of claim 1, further comprising a high frequency noise detector in data communication with the microcontroller.

7. The electronic circuit of claim 6, wherein the logic module is configured to suspend re-writing of the configuration data when the high frequency noise detector indicates a presence of noise, and is configured to commence the re-writing of the configuration data only after a predefined time period without noise.

8. The electronic circuit of claim 1, wherein the configuration data comprises values indicative of electrode input multiplexing, channel amplifier gain, and channel filter frequency.

9. The electronic circuit of claim 1, wherein the switch matrix further comprises a digital control loop having an analog to digital converter (ADC) configured to digitize a signal at each channel of the plurality of channels of the amplifier to generate a first value indicative of an offset voltage potential, a digital to analog converter (DAC), and analog circuitry, wherein the ADC and DAC are in data communication with the microcontroller, and wherein the logic module further causes the microcontroller to:
receive the first value;
continue to increase a counter by incremental values until the first value exceeds a positive guard limit or continue to decrease the counter by decremented values until the first value exceeds a negative guard limit;
determine a modulus value of the incremented or decremented counter value;
adjust the DAC potential to a second value that is equal and opposite to the first value, if the modulus value exceeds an offset guard threshold; and
apply the second value of the DAC potential to said each channel in order to nullify an effect of the offset voltage potential.

10. The electronic circuit of claim 9, wherein the switch matrix comprises a first multiplexer configured to allow a first input on its output line and a second multiplexer configured to allow a second input on its output line.

11. The electronic circuit of claim 10, wherein the switch matrix further comprises a differential amplifier, and wherein the differential amplifier receives the first input and the second input in order to generate said each channel as a difference of the first input and the second input.

12. The electronic circuit of claim 9, wherein another adjustment of the DAC potential is suspended for a predefined period of time.

13. A method of automatically re-writing configuration data of a plurality of control lines of one or more multiplexers of a switch matrix module, wherein the switch matrix module is integrated into an amplifier of an intraoperative neurophysiological monitoring (IONM) system, and wherein the plurality of control lines configures a plurality of channels of the amplifier, the method comprising:
writing configuration data serially to a field programmable gate array (FPGA) having an input register and a comparator, wherein the comparator receives first data of the first output register and second data of the second output register, and wherein the FPGA is in data communication with a microcontroller and the plurality of control lines;
receiving an error flag from the comparator, wherein the error flag is indicative of the first data not matching the second data; and
re-writing, in response to the error flag, the configuration data to the FPGA.

14. The method of claim 13, wherein the FPGA further comprises a parallel bus latched at a readback register and at a first output register and a second output register.

15. The method of claim 14, further comprising converting serially written configuration data to the parallel bus.

16. The method of claim 14, wherein the readback register allows readback of the configuration data serially by the microcontroller.

17. The method of claim 14, wherein the first output register is connected to external pins used as the plurality of control lines.

18. The method of claim 17, wherein the second output register is not connected to any external pins.

19. The method of claim 13, wherein the switch matrix module further comprises a high frequency noise detector in data communication with the microcontroller.

20. The method of claim 19, wherein the re-writing of the configuration data is suspended when the high frequency noise detector indicates a presence of noise, and wherein the re-writing commences only after a predefined time period of no noise.

21. The method of claim 13, wherein the configuration data comprises values indicative of electrode input multiplexing, channel amplifier gain, and channel filter frequency.

22. The method of claim 13, wherein the switch matrix module further comprises a digital control loop having an analog to digital converter (ADC) configured to digitize a signal at each channel of the plurality of channels of the amplifier to generate a first value indicative of an offset voltage potential, a digital to analog converter (DAC), and analog circuitry, wherein the ADC and DAC are in data communication with the microcontroller, and wherein the method further comprises:
receiving, from the ADC, a first value indicative of an offset voltage potential at said each channel;
continuing to increase a counter by incremental values until the first value continues to exceed a positive guard limit or continuing to decrease the counter by decremented values until the first value continues exceed a negative guard limit;
determining a function of the incremented or decremented counter value;
adjusting the DAC potential to a second value that is equal and opposite to the first value, if an output of the function exceeds an offset guard threshold; and
applying the second value of the DAC potential to said each channel in order to nullify an effect of the offset voltage potential.

23. The method of claim 22, wherein the switch matrix module comprises a first multiplexer configured to allow a first input on its output line and a second multiplexer configured to allow a second input on its output line.

24. The method of claim 22, wherein the switch matrix module further comprises a differential amplifier and wherein the differential amplifier receives the first and second inputs in order to generate said each channel as a difference of the first and second inputs.

25. The method of claim 22, wherein another adjustment of the DAC potential is suspended for a predefined period of time.

26. The method of claim 22, wherein the function is a modulus value.

27. An intraoperative neurophysiological monitoring (IONM) system comprising:
a base module;
a power module, wherein the base module is electrically coupled to a computing device through the power module;
an auditory and visual stimulator (AVX) module in electrical communication with the base module;
at least one electrical stimulator in electrical communication with the AVX module and with the base module;
at least one amplifier module in electrical communication with the base module; and
at least one transcranial stimulator (TCS) extender in electrical communication with the base module.

28. The intraoperative neurophysiological monitoring (IONM) system of claim 27, further comprising an identification (ID) chip in a connector at a patient end of a cable connected to the at least one amplifier module.

29. The intraoperative neurophysiological monitoring (IONM) system of claim 27, further comprising an identification (ID) chip in a connector at a patient end of a cable connected to the at least one TCS extender.
